SEQUENCE LISTING (5 sequences)

SEQ ID NO: 1 - protein, 75 aa, Artificial Sequence [sequence omitted]

SEQ ID NO: 2 - protein, 97 aa, Artificial Sequence
  Synthetic polypeptide
  MISC_FEATURE (1)..(10): Optional residues
  MISC_FEATURE (11)..(21): Optional residues
MGSSHHHHHHSSGLVPRGSHMGDAQDKLKYLVKQLERALRELKKSLDELERSLEELEKNPSEDALVENNRLNVENNKIIVEVLRIILELAKASAKLA

SEQ ID NO: 3 - protein, 87 aa, Artificial Sequence
  Synthetic polypeptide
  MISC_FEATURE (1)..(12): Optional residues
SSGLVPRGSHMGDAQDKLKYLVKQLERALRELKKSLDELERSLEELEKNPSEDALVENNRLNVENNKIIVEVLRIILELAKASAKLA

SEQ ID NO: 4 - protein, 87 aa, Artificial Sequence; identical to SEQ ID NO: 3
  Synthetic polypeptide
  MISC_FEATURE (1)..(7): Optional residues

SEQ ID NO: 5 - protein, 80 aa, Artificial Sequence
  Synthetic polypeptide
GSHMGDAQDKLKYLVKQLERALRELKKSLDELERSLEELEKNPSEDALVENNRLNVENNKIIVEVLRIILELAKASAKLA

(19) United States
(12) Patent Application Publication
(10) Pub. No.: US 2022/0185853 A1
(43) Pub. Date: Jun. 16, 2022

(54) AMANTADINE BINDING PROTEIN

(71) Applicant: UNIVERSITY OF WASHINGTON, Seattle, WA (US)

(72) Inventors: Jooyoung PARK, Seattle, WA (US); Scott BOYKEN, Seattle, WA (US); Kathy WEI, Seattle, WA (US); Gustav OBERDORFER, Seattle, WA (US); David BAKER, Seattle, WA (US)

(21) Appl. No.: 17/603,080

(22) PCT Filed: Apr. 15, 2020
(86) PCT No.: PCT/US2020/028280

§ 371 Date: Oct. 12, 2021

Related U.S. Application Data
(60) Provisional application No. 62/834,592, filed on Apr. 16, 2019.

(51) Int. Cl. C07K 14/47; C12N 15/63

(52) U.S. Cl. CPC C07K 14/47; A61K 38/00; C12N 15/63

(57) ABSTRACT

Disclosed herein are amantadine binding polypeptides, fusion proteins thereof, and uses of such polypeptides and fusion proteins.

### CROSS REFERENCE

This application claims priority to U.S. Provisional Patent Application Ser. No. 62/834592 filed Apr. 16, 2019, incorporated by reference herein in its entirety.

### BACKGROUND

No chemically-inducible trimerization systems have been developed despite the importance of trimerization in pro-apoptotic and pro-inflammatory signaling cascades. The design of a small molecule-inducible trimerizer is hence a challenge for de novo protein design with considerable practical relevance.

### REFERENCE TO SEQUENCE LISTING

This application contains a Sequence Listing submitted as an electronic text file named “19-142-PCT_Sequence-Listing_ST25.txt”, having a size in bytes of 5 kb, and created on Apr. 8, 2020. The information contained in this electronic file is hereby incorporated by reference in its entirety pursuant to 37 CFR § 1.52(e)(5).

### SUMMARY

In one aspect, the disclosure provides polypeptide comprising an amino acid sequence at least 75%, 80%, 85%, 90%, 91%, 92%, 93%, 94%, 95%, 96%, 97%, 98%, 99%, or 100% identical along the full length of the amino acid sequence of SEQ ID NO:1, wherein the polypeptide includes a residue selected from the group consisting of S71 and T71 at position 71 based on the numbering of residues in SEQ ID NO:1. In one embodiment, the polypeptide includes a hydrophobic residue at each of positions 64, 67, and 68 based on the numbering of residues in SEQ ID NO:1. In another embodiment, the polypeptide includes an alanine residue at one or more of positions 64, 67, and 68 based on the numbering of residues in SEQ ID NO:1. In a farther embodiment, the polypeptide includes an alanine residue at one or more of positions 67 and 68 based on the numbering of residues in SEQ ID NO:1. In one embodiment, 164, L67, 68, and S71 residues based on the numbering of residues in SEQ ID NO:1 are conserved in the polypeptide. In various embodiments, the polypeptide comprises an amino acid sequence at least 75%, 80%, 85%, 90%, 91%, 92%, 93%, 94%, 95%, 96%, 97%, 98%, 99%, or 100% identical along the full length of the amino acid sequence of the amino acid sequence of SEQ ID NO:2, SEQ ID NO:3, SEQ ID NO:4, or SEQ ID NO:5, wherein residues in parentheses are optional. In another embodiment, residue 6L relative to the sequence of SEQ ID NO:1 is modified to 6Q.

In one embodiment, each of residues 16, 17, 20, 24, 27, 31, 41, 42, 43, 49, 51, 56, 57, 58, 59, and 60 relative to SEQ ID NO:1 are hydrophobic residues. In another embodiment, 1, 2, 3, 4, 5, 6, 7, 8, 9, 10, 11, 12, 13, 14, 15, or all 16 of the following residues are conserved relative to SEQ ID NO:1: A16, L17, L20, L24, L27, L31, A41, L42, V43, L49, V51, 156,157, V58, V59, L60. In a further embodiment, each of residues 30, 46, 47, 50, 23, 53, and 54 relative to SEQ ID NO:1 are hydrophilic residues. In another embodiment, 1, 2, 3, 4, 5, 6, or all 7 of the following residues are conserved relative to SEQ ID NO:1: S30, N46, N47, N50, S23, N53, and N54. In one embodiment, 1, 2, 3, 4, 5, 6, 7, 8, 9, 10, 11, 12, 13, 14, 15, 16, 17, 18, 19, 20, 21, 22, or all 23 of the following residues are conserved relative to SEQ ID NO:1: A16, L17, L20, L24, L27, L31, A41, L42, V43, L49, V51, 156, 157, V58, V59, L60, S30, N46, N47, N50, S23, N53, and N54. In another embodiment, amino acid changes from the reference protein (SEQ ID NO:1) are conservative amino acid substitutions.

In one embodiment, the disclosure provides fusion proteins, comprising the polypeptide of any embodiment or combination of embodiments of the disclosure genetically fused to a bioactive polypeptide, including but not limited to a cell death polypeptide such as caspases−1, −3, −8, or −9.

In another embodiment, the disclosure provides polypeptides or fusion proteins of any embodiment or combination of embodiments of the disclosure, bound to amantadine. In one embodiment, the polypeptide or fusion protein is a monomer or a homo-trimer. In another embodiment, the disclosure provides polypeptides or fusion proteins of any embodiment or combination of embodiments of the disclosure bound to or embedded within a lipid membrane.

The disclosure also provides nucleic acid encoding the polypeptide or fusion protein of any embodiment or combination of embodiments of the disclosure, expression vectors comprising the nucleic acid operably linked to a suitable control element, and host cells comprising the nucleic acid claim, expression vector, polypeptides, or fusion proteins of any embodiment or combination of embodiments of the disclosure. The disclosure also provides pharmaceutical composition comprising the polypeptide, fusion protein, nucleic acid, expression vector, and/or host cell of any embodiment or combination of embodiments of the disclosure, and a pharmaceutically acceptable carrier. The disclosure also provides methods for using the polypeptides, fusion proteins, nucleic acids, expression vectors, host cells, or pharmaceutical compositions of any embodiment or combination of embodiments of the disclosure for any suitable purpose, including but not limited to as a safety switch for cell or gene therapy.

### DESCRIPTION OF THE FIGURES

FIG. 1a-c. Computational design methodology. (a) The homo-trimeric scaffold was designed to bind amantadine such that the C3 axes of the protein and the small molecule are aligned. (b) The binding pocket in ABP was designed to have polar serine residues (Ser-71) that hydrogen-bond (dashed lines) to the amino group of amantadine and nonpolar residues (Ile-64, Leu-67, and Ala-68) to complement the shape of the hydrophobic moiety of amantadine. (c) The design model contains hydrogen-bond networks that specify the trimeric assembly of ABP.

FIG. 2a-c. Binding characterization of amantadine to ABP. (a) SEC chromatogram monitoring absorbance at 280 nm (mAU) and estimated molecular mass (from MALS). (b) Apo-ABP (open circle) exhibits a high initial fluorescence signal that is lowered in the presence of amantadine (solid circle). As expected, 2LC3H6_13 (open diamond) and 2LC3H6_13 plus amantadine (solid diamond) exhibit a very low initial fluorescence signal. (c) The CD spectrum of ABP at 25° C., 75° C., 95° C., and 25° C. after heating and cooling. The CD spectrum of ABP at 25° C. suggests an all a-helical structure that remains fairly stable up to 75° C.

FIG. 3a-d. Structural characterization of the ABP-amantadine interaction. (a) The high-resolution X-ray structure (white) of ABP in complex with amantadine are very close to the computational model (gray) (RMSD of 0.63 Å and 0.59 Å, respectively). (b) Positive electron density corresponding to amantadine can be observed within the binding site of ABP prior to modeling in the ligand (Fₒ-F_{c} map contoured at 3.0σ). (c) Addition of the ligand in model building and refinement results in clear observable electron density corresponding to amantadine (2Fₒ-F_{c} map contoured at 1.0σ). (d) Clear electron density can be observed for amantadine and ordered water molecules in the binding site of ABP (2Fₒ-F_{c}map contoured at 1.0σ). Water-mediated hydrogen bonds are observed between Ser-71 and the amino group of amantadine (black dashed lines).

FIG. 4. CD spectrum of ABP in the presence amantadine. The CD spectrum of ABP in the presence of 5 mM amantadine at 25° C., 75° C., 95° C., and 25° C. after heating and cooling suggests that the thermal stability of ABP is not significantly affected by the presence of amantadine.

FIG. 5. Stereo images of the electron density map for a representative region of ABP. The 2Fₒ-F_{c} electron density map contoured at 1.0Υ.

FIG. 6. Representative thermofluor melting curve for ABP_L6Q. ABP_L6Q—like ABP—exhibits a high initial fluorescence signal (clear circle) that is lowered in the presence of amantadine (black circle).

FIG. 7. X-ray crystal structure of ABP_L6Q in complex with amantadine. The X-ray crystal structure of ABP_L6Q+amantadine (2.00 Å) is very similar to the ABP+amantadine structure. Crystallographic water molecules are shown as spheres.

### DETAILED DESCRIPTION

All references cited are herein incorporated by reference in their entirety. As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise fonn disclosed. While the specific embodiments of, and examples for, the disclosure are described herein for illustrative purposes, various equivalent modifications are possible within the scope of the disclosure, as those skilled in the relevant art will recognize.

In one aspect the disclosure provides polypeptides comprising an amino acid sequence at least 75%, 80%, 85%, 90%, 91%, 92%, 93%, 94%, 95%, 96%, 97%, 98%, 99%, or 100% identical along the full length of the amino acid sequence of SEQ ID NO:1, wherein the polypeptide includes a residue selected from the group consisting of S71 and T71 at position 71 based on the numbering of residues in SEQ ID NO:1.

>ABP_designedsequence
(SEQ ID NO: 1)
DAQDKLKYLVKQLERALRELKKSLDELERSLEELEKNPSEDALVENNRLN
VENNKIIVEVLRIILELAKASAKLA

As shown in the examples herein, the inventors have demonstrated that the polypeptides disclosed herein are capable of binding to amantadine and thus can be used, for example, as a safety switch for cell or gene therapy. For example, the polypeptides can be linked to cell death proteins (pro-apoptosis proteins, etc.) and expressed in cells being used for cell therapy; amantadine can then be administered to the subject to promote cell death of the cells used for cell therapy. The polypeptides disclosed herein constitute the first successful de novo design of a homo-trimeric protein that binds a C₃ symmetric small molecule.

In one embodiment, the polypeptide includes a hydrophobic residue at positions 64, 67, and 68 based on the numbering of residues in SEQ ID NO:1. Hydrophobic residues are defined herein as Ala, Cys, Gly, Pro, Met, Sce, Sme, Val, Ile, and Leu. In another embodiment, the polypeptide includes an alanine residue at one or more of positions 64, 67, and 68 based on the numbering of residues in SEQ ID NO:1. In a further embodiment, the polypeptide includes an alanine residue at one or more of positions 67 and 68 based on the numbering of residues in SEQ ID NO:1. In a still further embodiment, residues 164, L67, A68, and S71, based on the numbering of residues in SEQ ID NO:1, are conserved in the polypeptide. Positions 64, 67, 68, and 71 are present at the amantadine binding interface. As used herein, “conserved” means identical.

In one embodiment, the polypeptides comprising an amino acid sequence at least 75%, 80%, 85%, 90%, 91%, 92%, 93%, 94%, 95%, 96%, 97%, 98%, 99%, or 100% identical along the full length of the amino acid sequence of the amino acid sequence of SEQ ID NO:2, wherein the residues in parentheses are optional.

SEQ ID NO: 2
(MGSSHHHHHH) (SSGLVPRGSHMG)DAQDKLKYLVKQLERALRELKKS
LDELERSLEELEKNPSEDALVENNRLNVENNKIIVEVLRIILELAKASAK
LA (ABP_full_ORFsequence)

In one embodiment, the polypeptides comprising an amino acid sequence at least 75%, 80%, 85%, 90%, 91%, 92%, 93%, 94%, 95%, 96%, 97%, 98%, 99%, or 100% identical along the full length of the amino acid sequence of the amino acid sequence of SEQ ID NO:3 SEQ ID NO:4, or SEQ ID NO:5, wherein the residues in parentheses are optional.

SEQ ID NO: 3
(SSGLVPRGSHMG)DAQDKLKYLVKQLERALRELKKSLDELERSLEELE
KNPSEDALVENNRLNVENNKIIVEVLRIILELAKASAKLA
(ABP_full_ORFsequence including some optional
residues)
SEQ ID NO: 4
(SSGLVPR)GSHMGDAQDKLKYLVKQLERALRELKKSLDELERSLEELE
KNPSEDALVENNRLNVENNKIIVEVLRIILELAKASAKLA
(ABP_full_ORFsequence_including some optional
residues)
SEQ ID NO: 5
GSHMGDAQDKLKYLVKQLERALRELKKSLDELERSLEELEKNPSEDALVE
NNRLNVENNKIIVEVLRIILELAKASAKLA
(ABP_full_ORFsequence)

In one embodiment, residue 6L (relative to SEQ ID NO:1) may be modified to 6Q, as described in the examples that follow. In another embodiment, each of residues 16, 17, 20, 24. 27, 31, 41, 42, 43, 49, 51, 56, 57, 58, 59, and 60 are hydrophobic residues. These residues are believed to be on the interior of the polypeptide and/or homotrimer thereof, and may be involved in homotrimer formation. In a further embodiment, 1, 2, 3, 4, 5, 6, 7, 8, 9, 10, 11, 12, 13, 14, 15, or all 16 of the following residues are conserved relative to SEQ ID

NO:1: A16, L17, L20, L24, L27, L31, A41, L42, V43, L49, V51, 156, 157, V58, V59, L60.

In one embodiment, each of residues 30, 46, 47, 50, 23, 53, and 54 are hydrophilic residues. These residues are believed to be on the interior of the polypeptide, and may be involved in hydrogen bond networks that contribute to homotrimer formation. In another embodiment, 1, 2, 3, 4, 5, 6, or all 7 of the following residues are conserved relative to SEQ ID NO:1:S30, N46, N47, N50, S23, N53, and N54.

In a further embodiment, 1 2, 3, 4, 5, 6, 7, 8, 9, 10, 11, 12, 13, 14, 15, 16, 17, 18, 19, 20, 21, 22, or all 23 of the following residues are conserved relative to SEQ ID NO:1: A16,L17,L20,L24, L27, L31, A41, L42, V43, L49, V51, I56, I57, V58, V59, L60, S30, N46, N47, N50, S23, N53, and N54.

In another embodiment, amino acid changes from the reference protein are conservative amino acid substitutions.

As used here, “conservative amino acid substitution” means that
hydrophobic amino acids (Ala, Cys, Gly, Pro, Met, Sce, Sme, Val, Ile, Leu) can only be substituted with other hydrophobic amino acids;
hydrophobic amino acids with bulky side chains (Phe, Tyr, Trp) can only be substituted with other hydrophobic amino acids with bulky side chains;
amino acids with positively charged side chains (Arg, His, Lys) can only be substituted with other amino acids with positively charged side chains;
amino acids with negatively charged side chains (Asp, Glu) can only be substituted with other amino acids with negatively charged side chains; and
amino acids with polar uncharged side chains (Ser, Thr, Asn, Gln) can only be substituted with other amino acids with polar uncharged side chains.

In another embodiment, the disclosure provides fusion proteins, comprising the polypeptide of any embodiment or combination of embodiments disclosed herein genetically fused to a bioactive polypeptide, including but not limited to a cell death polypeptide such as caspases−1, −3, −8, or −9. A bioactive polypeptide is a polypeptide possessing any activity suitable for an intended purpose. In one non-limiting example, the bioactive polypeptide may comprise a cell death polypeptide. Any suitable cell death polypeptide may be linked to the polypeptides of the disclosure, including but not limited to caspases. The polypeptides disclosed herein are capable of binding to amantadine. Thus, for example, the polypeptides can be expressed in cells being used for cell therapy; amantadine can then be administered to the subject to promote cell death of the cells used for cell therapy as deemed appropriate by attending medical personnel. The polypeptides of the disclosure and the bioactive polypeptide may be linked by an amino acid linker of any suitable length or amino acid composition, as deemed appropriate for an intended use.

In one embodiment, the polypeptides or fusion proteins of any embodiment or combination of embodiments herein, are bound to or embedded within a lipid membrane. In one such embodiment, the polypeptides or fusion proteins are expressed on the surface of a cell. This embodiment may be used for cell therapy as discussed above.

In another embodiment, the disclosure provides polypeptides or fusion proteins of any embodiment or combination of embodiments disclosed herein, wherein the polypeptide or fusion protein is a monomer or a homo-trimer. As described in the examples, the polypeptides of the disclosure bind amantadine and can form homo-trimers.

In another embodiment, the disclosure provides homo-trimeric polypeptides or fusion proteins of any embodiment or combination of embodiments disclosed herein, bound to amantadine. Such binding complexes may be formed, for example, in the course of cell therapy as discussed above. Binding characteristics and assays for detecting such binding are exemplified in detail in the attached examples. In various non-limiting embodiments, detection of binding may be carried out by differential scanning fluorimetry, nuclear magnetic resonance, X-ray and neutron scattering studies. In another aspect the disclosure provides nucleic acids encoding the polypeptide or fusion protein of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides or fusion proteins of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence.

Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the polypeptides, fusion proteins, nucleic acids, expression vectors (i.e.: episomal or chromosomally integrated), polypeptides, or fusion proteins disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection. In one embodiment, the host cells express the polypeptides or fusion proteins on the cell surface.

In another aspect, the disclosure provides pharmaceutical compositions comprising the polypeptide, fusion protein, nucleic acid, expression vector, and/or the host cell of any embodiment or combination of embodiments disclosed herein, and a pharmaceutically acceptable carrier. The pharmaceutical compositions of the disclosure can be used, for example, in the methods of the disclosure described below. The pharmaceutical composition may comprise in addition to the polypeptide of the disclosure (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The pharmaceutical composition may also include a lyoprotectant, e.g.

sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenyhnercuric nitrate, thimerosal, benzoic acid, and various mixtures thereof In other embodiments, the pharmaceutical composition includes a bulking agent, like glycine.

In yet other embodiments, the pharmaceutical composition includes a surfactant e.g., polysorbate-20, polysorbate-40, polysorbate- 60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptides, fusion proteins, nucleic acids, expression vectors, and/or host cells may be the sole active agent in the pharmaceutical composition, or the composition may further comprise one or more other active agents suitable for an intended use. The polypeptides, fusion proteins, nucleic acids, expression vectors, host cells, and pharmaceutical compositions of the disclosure may be used for any suitable purpose, as described in detail herein.

In another aspect, the disclosure provides uses of the polypeptides, fusion proteins, nucleic acids, expression vectors, host cells, or pharmaceutical compositions disclosed herein for any suitable purpose, including but not limited to as a safety switch for cell or gene therapy.

As shown in the examples herein, the inventors have demonstrated that the polypeptides disclosed herein are capable of binding to amantadine and thus can be used, for example, as a safety switch for cell or gene therapy. For example, the polypeptides can be linked to cell death proteins (pro-apoptosis proteins, etc.) and expressed in cells being used for cell therapy; amantadine can then be administered to the subject to promote cell death of the cells used for cell therapy. In one embodiment, the polypeptides or fusion proteins are present on the cell surface.

### EXAMPLES

We used de novo protein design to create a homo-trimeric protein that binds the small molecule drug amantadine. The X-ray structure is very close to the design model, the neutron structure recapitulates the designed hydrogen-bond networks (data not shown), and solution NMR data show that amantadine-binding induces localized structural changes (data not shown). Small molecule-binding at a C₃ symmetric protein interface is an advance for computational protein design.

No chemically-inducible trimerization systems have been developed despite the importance of trimerization in pro-apoptotic and pro-inflammatory signaling cascades. The design of a small molecule-inducible trimerizer is hence a challenge for de novo protein design with considerable practical relevance.

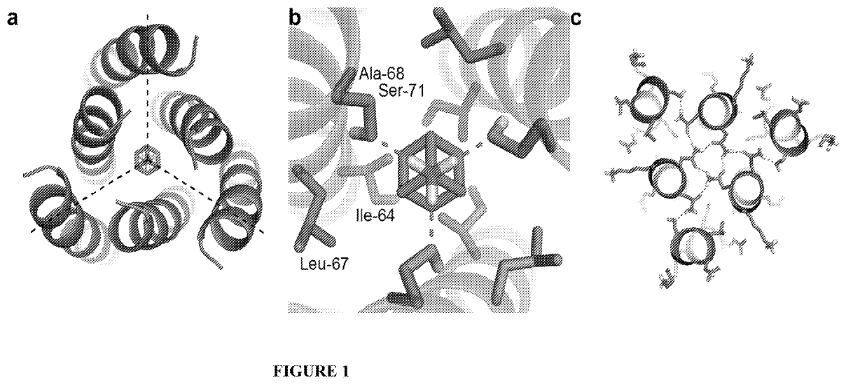

We set out to design trimeric proteins that bind small molecules with three-fold symmetry on their symmetry axes. We focused on the C₃ symmetric compound amantadine as it is an FDA approved drug with a low side effect profile⁹. To de novo design amantadine-binding sites at the protein trimer C₃ axes, we started from parametrically generated C₃ symmetric helical bundle backbones consisting of two concentric rings each with three helices. The symmetry axes of the protein scaffold and the amantadine were aligned, and the remaining two degrees of freedom (the placement along the symmetry axis, and the rotation around this axis) were sampled by grid search (FIG. 1a). For each placement, RosettaDesign™ was used to optimize the identities and conformations of the residues within 12.5 Å of the amantadine for high affinity binding, and residue conformations distances farther than 12.5 Å to retain hydrogen-bond networks identified by Rosetta HBNet™ (FIG. 1b-c). We found a particularly low energy solution starting from a previously characterized design with a high-resolution crystal structure (2L6HC3_13)¹⁰. (FIG. 1a). This solution, which we refer to as ABP (amantadine-binding protein), contains hydrogen bonds from Ser-71 to the polar amino group of amantadine and a shape complementary binding pocket composed by Ile-64, Leu-67, and Ala-68 (FIG. 1b).

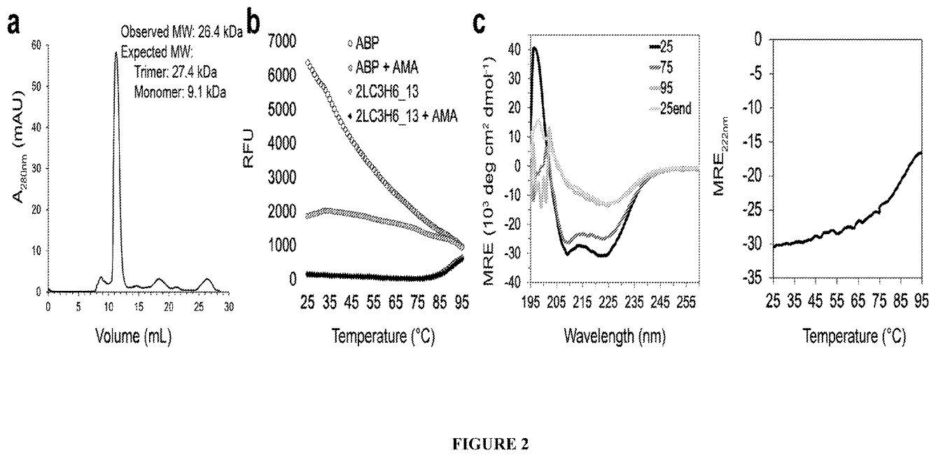

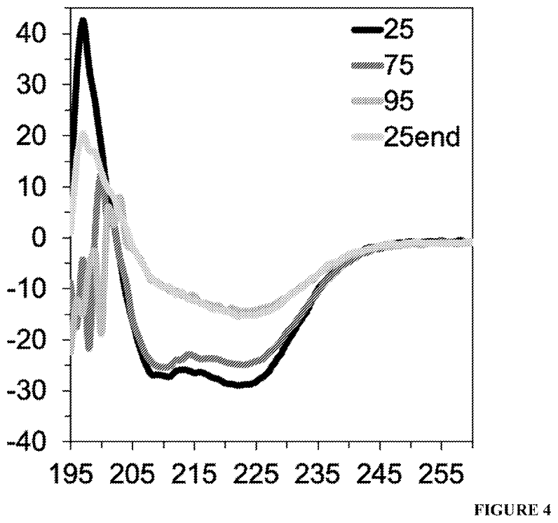

A synthetic gene encoding ABP was obtained and the protein expressed in E. coli.The design was expressed at high levels in the soluble fraction and was found by SEC-MALS to be a trimer in the presence and absence of amantadine (FIG. 2a). Interactions with amantadine were probed using thermofluor dye binding assay (differential scanning fluorimetry). The thermofluor melting curve for apo-ABP exhibited a high initial fluorescence signal at 25° C. (FIG. 2b), indicating that hydrophobic residues in the protein core are exposed to solvent. As the protein was heated to 95° C., the fluorescence signal decreased, corresponding to protein aggregation at higher temperatures. In the presence of amantadine (1mM), the initial fluorescence signal was much lower, characteristic of properly folded proteins (FIG. 2b), suggesting that amantadine binding may cause local ordering and exclude solvent. In contrast, 2L6HC3_13, which has the same backbone parameters but lacks the amantadine binding site, is thermally stable by thermofluor assay, only starting to denature at ˜80° C. (FIG. 2b). As expected, amantadine had no effect on the melting curve of 2L6HC3_13, suggesting the interactions with ABP are through the designed binding site (FIG. 2b). The CD spectrum of ABP at 25° C. suggests an all a-helical structure, with negative bands at 222 nm and 208 nm, and a positive band at 190 nm (FIG. 2c). As the sample was heated to 95° C., a loss in CD signal was observed which was not significantly altered in the presence of 1 mM amantadine (FIG. 2c FIG. 4).

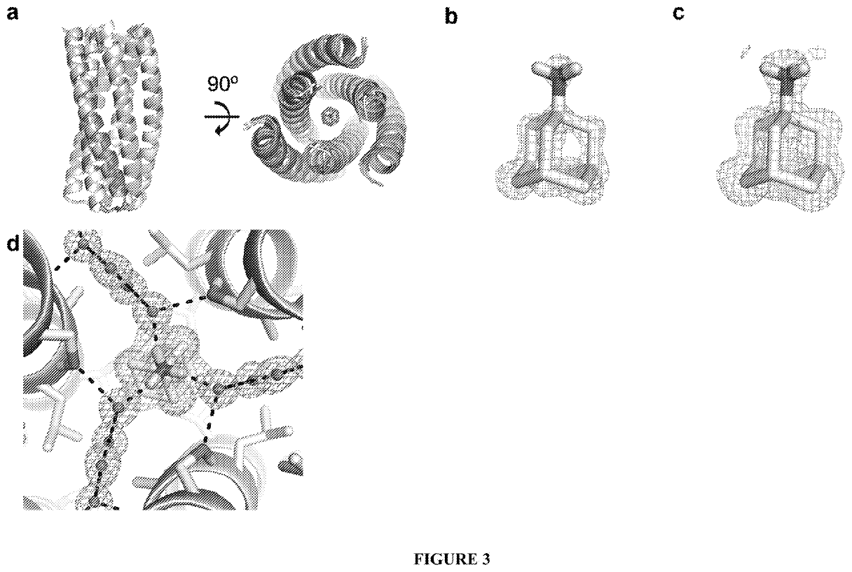

We carried out crystallographic studies to characterize the interaction between ABP and amantadine. Crystallization screen trays were set up with the same protein sample with or without ˜five-fold molar excess amantadine (7.5 mM). Crystals were obtained in the presence but not the absence of amantadine, consistent with ordering upon amantadine binding. The X-ray crystal structure of ABP+amantadine was solved to 1.04 Å, providing a high-resolution view of the ABP-amantadine complex structure (FIG. 3a). The crystal structure overlays well with the design model, with an RMSD of 0.63 A (TMAlign¹¹) (FIG. 3a). The primary difference between the design model and crystal structure is in the compactness of helices in the amantadine-binding region (FIG. 3a). Clear electron density was observed for amantadine with ordered water molecules that mediate hydrogen bonding to Ser-71 residues in ABP (FIG. 3b-d).

Our results are an advance for protein design as to our knowledge this is the first successful de novo design of a homo-trimeric protein that binds a C3 symmetric small molecule. The designed protein contains hydrogen-bond networks that specify the trimeric state and water-mediated binding to amantadine. The solution NMR data (data not shown) suggest that ABP adopts a stable, symmetric structure and readily binds amantadine. The high-resolution X-ray crystal structure of the designed protein in complex with amantadine is very close to the computational model, and the neutron structure (data not shown) demonstrates the presence of the designed hydrogen-bond networks.

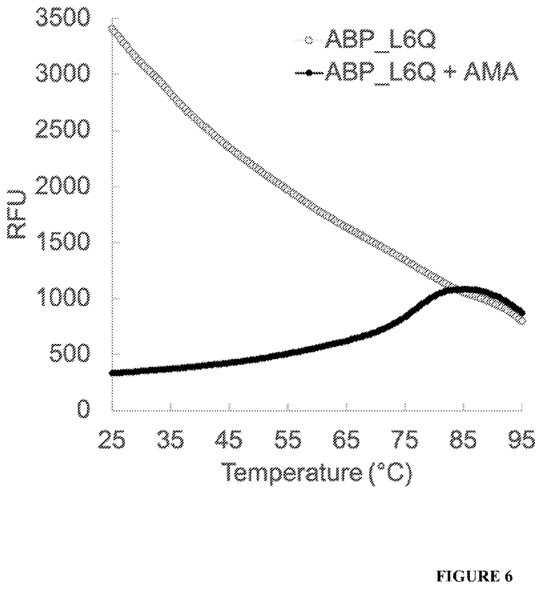

A mutant variant of ABP−ABP_L6Q—was expressed and purified in the same manner as described for ABP·ABP_L6Q exhibited a similar profile to ABP by thermofluor assay (FIG. 6). Like ABP, the thermofluor melting curve for apo-ABP_L6Q exhibited a high initial fluorescence signal at 25° C., indicating that hydrophobic residues in the protein core are exposed to solvent. As the protein was heated to 95° C., the fluorescence signal decreased, corresponding to protein aggregation at higher temperatures. In the presence of amantadine (1mM), the initial fluorescence signal was much lower, characteristic of properly folded proteins, suggesting that amantadine binding may cause local ordering and exclude solvent (FIG. 6).

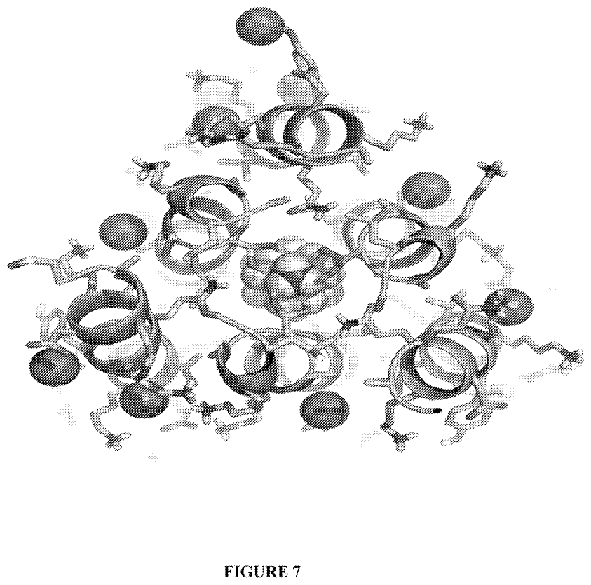

Crystallization screen trays were set up with ABP_L6Q in the presence of five-fold molar excess amantadine (7.5 mM). The X-ray crystal structure of ABP_L6Q+amantadine was solved to 2.00 Å (FIG. 7). Two alternate conformations of the Ser-71 residues were observed: one set of conformers making hydrogen bond interactions with amantadine, and another set where the Ser-71 residues now make sub-optimal hydrogen bonding to Q6 in this mutant.

### REFERENCES

1. Spencer, D. M. et al. Functional analysis of Fas signaling in vivo using synthetic inducers of dimerization. Curr. Biol. 6, 839-847 (1996).
2. Spencer, D. M., Wandless, T. J., Schreiber, S. L. & Crabtree, G. R. Controlling signal transduction with synthetic ligands. Science 262, 1019-1024 (1993).
3. Clackson, T. et al. Redesigning an FKBP-ligand interface to generate chemical dimerizers with novel specificity.
4. Mallet, V. O. et al. Conditional cell ablation by tight control of caspase-3 dimerization in transgenic mice. Nat. Biotechnol. 20, 1234-1239 (2002).
5. Guerrero, A. D., Chen, M. & Wang, J. Delineation of the caspase-9 signaling cascade. Apoptosis 13, 177-186 (2008).
6. Nyanguile, O., Uesugi, M., Austin, D. J. & Verdin, G. L. A nonnatural transcriptional coactivator. Proc. Natl. Acad. Sci. U. S. A. 94, 13402-13406 (1997).
7. Stankunas, K. et al. Conditional Protein Alleles Technique Using Knockin Mice and a Chemical Inducer of Dimerization. Mol. Cell 12, 1615-1624 (2003).
8. Miyamoto, T. et al. Rapid and orthogonal logic gating with a gibberellin-induced dimerization system. Nat. Chem. Biol. 8, 465-470 (2012).
9. Perez-Lloret, S. & Rascol, O. Efficacy and safety of amantadine for the treatment of L-DOPA-induced dyskinesia. J. Neural Transm. 125, 1237-1250 (2018).
10. Boyken, S. E. et al. De novo design of protein homo-oligomers with modular hydrogen-bond network-mediated specificity. Science 352, 680-687 (2016).
11. Zhang, Y. & Skolnick, J. TM-align: a protein structure alignment algorithm based on the TM-score. Nucleic Acids Res. 33, 2302-2309 (2005).
12. Thomaston, J. L. et al. Inhibitors of the M2 Proton Channel Engage and Disrupt Transmembrane Networks of Hydrogen-Bonded Waters. J. Am. Chem. Soc. 140, 15219-15226 (2018).
13. Wang, J. et al. Molecular dynamics simulation directed rational design of inhibitors targeting drug-resistant mutants of influenza A virus M2. J. Am. Chem. Soc. 133, 12834-12841 (2011).

### Supplemental Methods:

### RosettaDesign™

Design calculations were performed using RosettaDesign™. The Rosetta™ software suite is available free of charge to academic users and can be downloaded from the Rosetta™ Commons web site.

The initial 2LC3H6_13 scaffold was previously generated using parametric design¹⁰. Briefly, the parametrically generated backbone was regularized using cartesian space minimization in Rosetta™ and a special instance of the HBNet™ protocol—HBNetStapleInterface™—was used to identify combinations of hydrogen-bond networks.

The helices of monomer subunits were connected into a single chain and the assembled proteins were designed using symmetric Rosetta™ sequence design calculations in C₃ symmetry.

In order to create the amantadine binding site, the RosettaScripts™ protocol was used with user-defined design of the residue positions within 15 Å of the ligand (.xml). A Rosetta™ constraint (.cst) file was used to specify the atom-pair constraints in amantadine. A molecule parameter (.params) file was generated for amantadine in RosettaDesign™. Amantadine was split into one third, and the nitrogen and carbon atoms on the axis of rotation were virtualized. Rotamers were repacked with LayerDesign™ and resfile types (nixes) were used to specify Ser/Thr at residue positions hydrogen-bonding to amantadine.

Cloning, protein expression and purification

ABP was cloned into the pET28b(+) vector at Ndel and XhoI restriction sites. Constructs were transformed into BL21-Star (DE3) competent cells (Life Technologies). Cells harboring the plasmid were grown at 37° C. in Terrific Broth™ II medium containing a final concentration of 0.05 mg/ml kanamycin. Once cells reached an OD600 of 0.6-0.8, cells were cooled to 18° C. and induced with 0.25 mM IPTG overnight. After this period, cells were harvested by centrifugation at 4,000 r.p.m. for 10 min at 4° C. Cell pellets were resuspended in 60 ml of 25 mM Tris (pH 8.0), 300 mM NaCl, 20 mM imidazole (pH 8.0), and 1 mM PMSF per 1 L of Terrific Broth™ II medium and stored at −80° C.

Cells were thawed in the presence of 0.25 mg/ml lysozyme and disrupted using sonication on ice for 60 s. The cell extract was obtained by centrifugation at 13,000 r.p.m. for 30 min at 4° C. and was applied onto Ni-NTA agarose beads (Qiagen) equilibrated with wash buffer (25 mM Tris (pH 8.0), 300 mM NaCl, and 20 mM imidazole (pH 8.0)). The wash buffer was used to wash the nickel column three times with five column volumes. After washing, protein was eluted with five column volumes of elution buffer (wash buffer with 300 mM imidazole).

The eluate was buffer-exchanged with SAXS buffer (25 nM Tris (pH 8.0), 150 mM NaCl, and 2% glycerol) to lower the imidazole concentration from ˜300 mM to <20 mM and cleaved with restriction-grade thrombin (EMD Millipore 69671-3) overnight at 20° C. After overnight cleavage, the sample was flowed over equilibrated Ni-NTA agarose beads and the flow-through was captured.

The protein sample was further purified by gel chromatography using a Superclex™ 75 Increase 10/300 GL column (GE Healthcare) equilibrated with SAXS buffer. The fractions containing the protein of interest were pooled and concentrated using a 3 K MWCO Amicon™ centrifugal filter (Millipore).

Thermofluor assay

Thermofluor assays were performed in SAXS buffer using a CFX96 Touch™ Real-Time PCR machine (Bio-Rad). Thermal stability assays were performed using 45 μL of 5 μM protein (with or without 1 mM amantadine) and 5 μL of freshly prepared 200X SYPRO™ orange (Thermo-Fisher) solution in SAXS buffer. The temperature was ramped from 25 ° C. to 95 ° C. in 0.5 ° C. increments with intervals of 5 s. Fluorescence was read in the FRET scanning mode. The average of three replicates of buffer+SYPRO orange solution (no protein control) was subtracted from the average of three replicates for each sample.

### Circular Dichroism

CD wavelength scans (260 to 195 nm) and temperature melts (25 to 95° C.) were measured using a JASCO™ J-1500 or an AVIV™ model 420 CD spectrometer. Temperature melts monitored absorption signal at 222 nm and were carried out at a heating rate of 4° C/min. Protein samples were prepared at 0.25 mg/mL in phosphate buffered saline (PBS) pH 7.4 in a 0.1 cm cuvette.

### Crystallization of ABP

Purified ABP sample was concentrated to approximately 13 mg/nal in SAXS buffer and incubated with 7.5 mM amantadine (˜five-fold molar excess). Samples were screened using the sparse matrix method (Jancarik and Kim, 1991) with a Phoenix Robot (Art Robbins Instruments, Sunnyvale, CA) utilizing the following crystallization screens: Morpheus

(Molecular Dimensions), JCSG+(Qiagen), and Index (Hampton Research). Crystals were obtained in crystallization condition JCSG+B9: 0.1 M Citric Acid (4.0), 20% w/v PEG 6000 (final pH 5.0). Crystals were obtained after 1 to 14 days by the sitting-drop vapor-diffusion method with the drops consisting of a 1:1 mixture of 0.2 μL protein solution and 0.2 μL reservoir solution.

### X-ray Diffraction Collection and Structure Determination of ABP

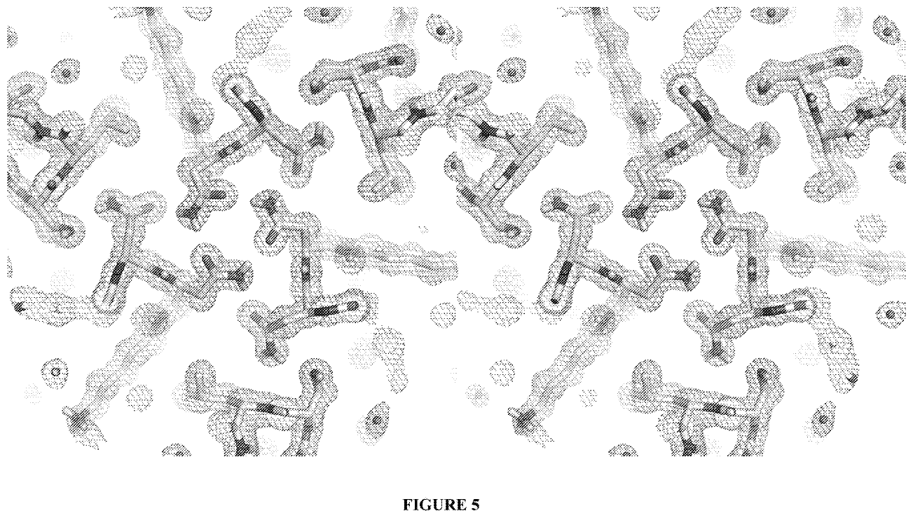

ABP crystals were placed in a reservoir solution containing 20% (v/v) glycerol, and then flash-cooled in liquid nitrogen. The X-ray data sets were collected at a wavelength of 1 Å at the Beamline 19-ID of the Advanced Photon Source (APS) at Argonne National Laboratory (ANL). Data sets were indexed and scaled using HKL2000¹⁸. All the design structures were determined by the molecular-replacement method with the program PHASER^{TM19} within the Phenix^{Tm} suite²⁰ using the design models as the initial search model. The atomic positions obtained from molecular replacement and the resulting electron density maps were used to build the design structures and initiate crystallographic refinement and model rebuilding. Structure refinement was performed using the phenix.refine²¹ program. Manual rebuilding using COOT²² and the addition of water molecules allowed construction of the final models. Root-mean-square deviation differences from ideal geometries for bond lengths, angles and dihedrals were calculated with Phenix^{TM}. The overall stereochemical quality of all final models was assessed using the program MOLPROBITY^{TM23}. The model showed 100% of the residues in favorable regions of the Ramachandran plot with 0% outliers. Figures were prepared with Pymol^{TM} (Pymol Molecular graphics System, Version 2.0; Schrodinger, LLC). A stereo image of a representative region of the electron density map is shown in FIG. 5.

We claim:

1. A polypeptide comprising an amino acid sequence at least 75%, 80%, 85%, 90%, 91%, 92%, 93%, 94%, 95%, 96%, 97%, 98%, 99%, or 100% identical along the full length of the amino acid sequence of SEQ ID NO:1, wherein the polypeptide includes a residue selected from the group consisting of S71 and T71 at position 71 based on the numbering of residues in SEQ ID NO:1.

2. The polypeptide of claim 1, wherein the polypeptide includes a hydrophobic residue at each of positions 64, 67, and 68 based on the numbering of residues in SEQ ID NO:1.

3. The polypeptide of claim 1 or 2, wherein the polypeptide includes an alanine residue at one or more of positions 64, 67, and 68 based on the numbering of residues in SEQ ID NO:1.

4. The polypeptide of claim 1 or 2, wherein the polypeptide includes an alanine residue at one or more of positions 67 and 68 based on the numbering of residues in SEQ ID NO:1.

5. The polypeptide of any one of claims 1-4, wherein 164, L67, A68, and S71 residues based on the numbering of residues in SEQ ID NO:1 are conserved in the polypeptide.

6. The polypeptide of any one of claims 1-5, comprising an amino acid sequence at least 75%, 80%, 85%, 90%, 91%, 92%, 93%, 94%, 95%, 96%, 97%, 98%, 99%, or 100% identical along the full length of the amino acid sequence of the amino acid sequence of SEQ ID NO:2, wherein the residues in parentheses are optional.
SEQ ID NO: 2
(MGSSHHHHHH) (SSGLVPRGSHMG)DAQDKLKYLVKQLERALRELKKS
LDELERSLEELEKNPSEDALVENNRLNVENNKIIVEVLRIILELAKASAK
LA (ABP_full_ORFsequence)

7. The polypeptide of any one of claims 1-5, comprising an amino acid sequence at least 75%, 80%, 85%, 90%, 91%, 92%, 93%, 94%, 95%, 96%, 97%, 98%, 99%, or 100% identical along the full length of the amino acid sequence of the amino acid sequence of SEQ ID NO:3, SEQ ID NO:4, or SEQ ID NO:5.

8. The polypeptide of any one of claims 1-7, wherein the polypeptide comprises an amino acid sequence at least 85% identical along the full length of the amino acid sequence of SEQ ID NO:1, SEQ ID NO:2, SEQ ID NO:3, SEQ ID NO:4, or SEQ ID NO:5.

9. The polypeptide of any one of claims 1-7, wherein the polypeptide comprises an amino acid sequence at least 90% identical along the full length of the amino acid sequence of SEQ ID NO:1, SEQ ID NO:2, SEQ ID NO:3, SEQ ID NO:4, or SEQ ID NO:5.

10. The polypeptide of any one of claims 1-7, wherein the polypeptide comprises an amino acid sequence at least 95% identical along the full length of the amino acid sequence of SEQ ID NO:1, SEQ ID NO:2, SEQ ID NO:3, SEQ ID NO:4, or SEQ ID NO:5.

11. The polypeptide of any one of claims 1-10, wherein residue 6L relative to the sequence of SEQ ID NO:1 is modified to 6Q.

12. The polypeptide of any one of claims 1-11, wherein each of residues 16, 17, 20, 24, 27, 31, 41, 42, 43 49, 51, 56, 57, 58, 59, and 60 relative to SEQ ID NO:1 are hydrophobic residues.

13. The polypeptide of any one of claims 1-12, wherein 1, 2, 3, 4, 5, 6, 7, 8, 9, 10, 11, 12, 13, 14, 15, or all 16 of the following residues are conserved relative to SEQ ID NO:1: A16, L17, L20, L24, L27, L31, A41, L42, V43, L49, V51, 156, 157, V58, V59, L60.

14. The polypeptide of any one of claims 1-13, wherein each of residues 30, 46, 47, 50, 23, 53, and 54 relative to SEQ ID NO:1 are hydrophilic residues.

15. The polypeptide of any one of claims 1-14, wherein 1, 2, 3, 4, 5, 6, or all 7 of the following residues are conserved relative to SEQ ID NO:1:S30, N46, N47, N50, S23, N53, and N54.

16. The polypeptide of any one of claims 1-15, wherein 1, 2, 3, 4, 5, 6, 7, 8, 9, 10, 11, 12, 13, 14, 15, 16, 17, 18, 19, 20, 21, 22, or all 23 of the following residues are conserved relative to SEQ ID NO:1: A16, L17, L20, L24, L27, L31, A41, L42, V43, L49, V51, 156, 157, V58, V59, L60, S30, N46, N47, N50, S23, N53. and N54.

17. The polypeptide of any one of claims 1-16, wherein amino acid changes from the reference protein are conservative amino acid substitutions.

18. A fusion protein, comprising the polypeptide of any one of claims 1-17 genetically fused to a bioactive polypeptide, including but not limited to a cell death polypeptide such as caspases−1, −3, −8, or −9.

19. The polypeptide of any one of claims 1-17, or the fusion protein of claim 18, bound to amantadine.

20. The polypeptide or fusion protein of any one of claims 1-19, wherein the polypeptide or fusion protein is a monomer or a homo-trimer.

21. The polypeptide or fusion protein of any one of claims 1-20, bound to or embedded within a lipid membrane.

22. A nucleic acid encoding the polypeptide or fusion protein of any one of claims 1-21.

23. An expression vector comprising the nucleic acid of claim 22 operably linked to a suitable control element.

24. A host cell comprising the polypeptide or fusion protein of any one of claims 1-21, the nucleic acid claim 22 and/or expression vector of claim 23.

25. A pharmaceutical composition comprising the polypeptide or fusion protein of any one of claims 1-21, the nucleic acid of claim 22, the expression vector of claim 23, and/or the host cell of claim 24, and a pharmaceutically acceptable carrier.

26. Use of the polypeptides, fusion proteins, nucleic acids, expression vectors, host cells, or pharmaceutical compositions of any of the preceding claims for any suitable purpose, including but not limited to as a safety switch for cell or gene therapy.
